(19) United States
(12) Patent Application Publication
(10) Pub. No.: US 2018/0364247 A1
(43) Pub. Date: Dec. 20, 2018

(54) DIAGNOSTIC KIT FOR CENTRAL NERVOUS SYSTEM AFFLICTIONS

(71) Applicant: Applied Nuerologics, LLC, W. Bloomfield, MI (US)

(72) Inventors: Mark Wasserman, Detroit, MI (US); Todd Flower, W. Bloomfield, MI (US); David Matzilevich, W. Bloomfield, MI (US)

(21) Appl. No.: 15/975,564

(22) Filed: May 9, 2018

Related U.S. Application Data
(63) Continuation of application No. 15/152,193, filed on May 11, 2016, now abandoned.

(51) Int. Cl. G01N 33/58; G01N 33/68; G01N 33/543

(52) U.S. Cl. CPC G01N 33/587; G01N 33/6896; G01N 2800/28; G01N 33/54386; G01N 2333/47; G01N 33/585

(57) ABSTRACT

A method and system for diagnosing concussions and other CNS afflictions is provided. A diagnostic kit includes a tube containing a predefined amount of lyophilized tau-specific antibody conjugated to colored latex nanoparticles, a fluid for mixing with the lyophilized tau-specific antibody conjugated to colored latex nanoparticles within the tube, so as to reconstitute the lyophilized tau-specific antibody conjugated to colored latex nanoparticles, resulting in a reconstituted mixture, and a swab comprising an absorbent hydrophobic material, the swab configured for absorbing cerebrospinal fluid when swabbed on a patient, wherein when the swab that has absorbed cerebrospinal fluid is placed in the tube containing the reconstituted mixture, the swab is configured to change color.

### CROSS-REFERENCE TO RELATED APPLICATIONS

This application is a continuation of patent application Ser. No. 15/152,193 filed May 11, 2016. The subject matter of patent application Ser. No. 15/152,193 is hereby incorporated by reference in its entirety

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The disclosed subject matter relates generally to the field of medicine and wellness, and, more specifically, to diagnoses for central nervous system afflictions.

### BACKGROUND

Traumatic brain injury (TBI) is the leading cause of death in the United States in people between the ages of 1 and 44 years and occurs in hundreds of thousands of subjects yearly. Recently, the importance of mild injuries has been recognized as a public health crisis for soldiers in the combat theater, children and young adults in sport activities, and other throughout their normal lives. Symptoms associated with TBI can appear immediately following injury or days or weeks later, and result in wide-ranging physical and psychological deficits including motor impairment, epilepsy, personality change and memory impairment.

Traumatic brain injuries (TBI) can occur in several ways and can be characterized by the mechanism of injury, the clinical severity as graded by the Glasgow Coma Scale (GCS), or by the characterization of the structural damage. Generally, injuries are classified as mild, moderate (both of which are considered concussion), or severe depending on the GCS score, which utilizes motor, eye and verbal responses to evaluate the level of cognition. In addition to the GCS score, TBI may be classified according to a clinical injury score based on the level of injury incurred to various key body regions, according to the level of structural damage incurred and according to the patient prognosis based on various prognostic models. Mild TBI is the most common sub category of TBI, with estimates ranging from 1.6 million to 3.8 million annually among athletes in the United States alone. Despite its designation, a mild TBI should not be viewed as an inconsequential injury, as some mild TBI can result in prolonged cognitive, emotional and functional disabilities, significantly impacting the quality of life.

TBI is heterozygous in nature, and no single classification is sufficient in characterizing injury for the purpose of diagnosis and prognosis. Based on the severity of the initial insult, different imaging techniques are used to obtain the necessary information for patient care. In cases of moderate to severe TBI, it is diagnosed through structural abnormalities using techniques such as computed tomography (CT) or conventional magnetic imaging (MRI) which is not very effective to diagnose these subtler irregularities. Mild TBI, such as concussion results in biochemical, metabolic and cellular changes that may be responsible for some long-term problems seen in patients who develop post-concussion syndrome (PCS).

Of all severities of Traumatic Brain Injury (TBI) an estimated 75-85% are categorized as mild TBI, which includes concussion as well as sub-concussion and some blast injuries associated with improvised explosive devices (IEDs). Concussion occurs in a wide variety of activities including sports, such as boxing, American football, rugby, soccer, cheerleading, ice hockey, wrestling, and military service; and in association with other exposures such as poorly controlled epilepsy and physical abuse. There is typically full neurologic recovery after a mild TBI; however, 15-30% of subjects develop prolonged neurocognitive and behavioral changes. Concussions are cumulative, and it is believed that the accumulation is one mechanism that can cause dementia.

It is estimated that 1.6-3.8 million concussions occur annually in the United States. Concussion is particularly frequent in American football, where 4.5% of high school, 6.3% of collegiate and 6.6% of professional football players are diagnosed with at least one concussion per season. In addition, the U.S. Department of Defense have diagnosed 339,462 concussions for U.S. Service members since 2000. The true frequency of concussion is probably much greater since concussions are typically unrecognized and sub-clinical, under-reported and can resolve spontaneously without medical care.

There is still no universal consensus regarding the definition of concussion. The 2013 Zurich Consensus Statement on Concussion in Sport proposed that concussion and mild TBI should be viewed as distinct entities. The group defined concussion as a “complex pathophysiological process affecting the brain” and allowed for the presence of neuropathological damage. However, concussive symptoms were largely thought to reflect a functional disturbance, typically resolving spontaneously with no imaging abnormality. In contrast, recent American Academy of Neurology guidelines for sport concussion in 2013 do not separate concussion from mild TBI, defining concussion as a “clinical syndrome of biomechanically induced alteration of brain function, typically affecting memory and orientation, which may involve loss of consciousness”. They noted, however, a lack of consensus in the use of the term, with an overlap in the use of concussion and mild TBI.

Therefore, concussion is currently used in two main ways: (1) to describe a distinct pathophysiological entity with its own diagnostic and management implications, mainly seen in the context of sporting injuries; and (2) to describe a constellation of symptoms that arise after different types of TBI.

There are also problems in retaining concussion as a diagnostic label for the constellation of symptoms that are commonly experienced after TBI. Concussion usually implies a “benign” set of problems that will eventually resolve spontaneously. However, the assumed transience of “concussion” symptoms is problematic, as many patients do not recover quickly and it is difficult to predict long-term outcome after TBI. Even apparently trivial injuries can sometimes have long-term effects with patients reporting similar post concussive symptoms after TBI of all severities.

Standard investigations also do not particularly help in defining “concussion”. Many patients with mild TBI will not undergo neuroimaging and are perhaps wrongly reassured about the concussive nature of their problems without any detailed investigation. Even when there is available neuroimaging, it is easy to be falsely reassured by negative neuroimaging findings. Standard neuroimaging will identify large focal contusions or hemorrhage but normal CT and MRI do not exclude diffuse axonal and vascular injury, both major drivers of poor clinical outcome after TBI. Standard neuroimaging sequences can miss these problems, although more advanced techniques such as susceptibility weighted and diffusion MRI are more sensitive and may identify them.

Blast-related TBI and concussion is among the most frequent injuries sustained by soldiers and other personnel who have served in recent wars in Iraq and Afghanistan. Difficulties of returning personnel with reintegrating into civilian society have been in part been attributed to brain injury that was caused by blast concussions. Reports of blast-related TBI in military personnel deployed to Iraq (Operation Iraqi Freedom) and Afghanistan (Operation Enduring Freedom) has been as high as 19%-23%. Reports of blast-related TBI among military personnel are unprecedented in comparison to military personnel in any other previous war or conflict. Moreover, blast concussion researchers do not enjoy the “unique advantages” that exist in conducting research in sports concussion samples. Like sports concussion samples soldiers and other military personnel represent a group that is at increased risk of sustaining mild TBI. However, blast concussions are only occasionally witnessed, assessments are not typically conducted in a standardized manner and a controlled environment just following the blast, and only rarely are personnel systematically followed during acute, subacute, and post-acute phases like in sports-related concussion.

Concerns have been noted about the reliability of retrospective self-report of blast events and TBI screening in military personnel. With few exceptions, evaluators of blast concussion are not able to corroborate acute-stage injury characteristics and cognitive performances immediately after blast concussions, which impede their ability to effectively diagnose blast-related concussions. Therefore, the evaluators of blast concussion confront a dilemma that can be solved by this diagnostic: blast concussion has been identified as a common hazard of the recent wars in Iraq and Afghanistan, but the elucidation and frequency associated with historical blast concussions is obscured by the unknown reliability and validity of self-report information obtained through contemporary TBI screening methods.

Mild TBI is usually caused by an impact to the head (contact loading) that induces rotational acceleration of the brain (inertial loading). In some patients, mild TBI occurs without an impact to the head such as after rapid rotational acceleration of the head in restrained occupants during a motor vehicle crash. At a neurophysiological level, these mechanical and inertial forces result in the stretching of white matter axons, leading to diffuse axonal injury.

It has long been understood that mild TBI, common in the sport of boxing can lead to dementia syndrome (dementia pugilistica) that includes Parkinson's disease-like motor signs and cognitive symptoms that include bradyphrenia (slowed thinking), confusion, and memory impairment. It is becoming evident that mild TBI experienced by football players is associated with chronic traumatic encephalopathy (CTE), a mid-life dementing disorder evidenced upon autopsy as prominent diffuse neurofibrillary tangles; a hallmark pathologic brain lesion observed in several other neurodegenerative diseases.

The underlying pathophysiology of mild TBI remains undetermined and as a result there is no efficient diagnostic, prognostic or therapeutic strategies currently available. Studies have begun to investigate mild TBI at the cellular and molecular level, as shortcomings in the current brain imaging techniques and flawed clinical diagnostic approaches have increased the appeal for a biochemical assay to diagnose mild TBI. For example, diagnosis and prognosis in moderate and severe TBI using conventional imaging techniques is informative. Specifically the goal of this approach is to uncover a single marker or panel of markers to aide in early detection and diagnosis, as well as potentially predict patient outcomes.

Consequently, there is an urgent medical need to develop a biochemical diagnostic for concussions. Currently, the only available TBI or concussion tests are either cognitive, which can be falsely interpreted, or require testing equipment that can only be found in hospitals or in testing laboratories. There is currently no non-invasive “field” concussion diagnostic that can be deployed on a sports field, in a school, workplace or battlefield. Therefore, a need exists to overcome the problems with the prior art as discussed above, and particularly for a more efficient and expeditious way of diagnosing concussions.

### SUMMARY

This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

A method and system for diagnosing concussions and other CNS afflictions is provided. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a diagnostic kit includes a tube containing a predefined amount of lyophilized tau-specific antibody conjugated to colored latex nanoparticles, a fluid for mixing with the lyophilized tau-specific antibody conjugated to colored latex nanoparticles within the tube, so as to reconstitute the lyophilized tau-specific antibody conjugated to colored latex nanoparticles, resulting in a reconstituted mixture, and a swab comprising an absorbent hydrophobic material, the swab configured for absorbing cerebrospinal fluid from a patient, wherein when the swab that has absorbed cerebrospinal fluid is placed in the tube containing the reconstituted mixture, the swab tip is configured to change color. In an additional embodiment, a method for diagnosing concussions and other CNS afflictions is also provided

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims. The foregoing and other features and advantages of the present invention will be apparent from the following more particular description of the preferred embodiments of the invention, as illustrated in the accompanying drawings.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1 is a flowchart showing the control flow of the process 100 for diagnosing a concussion, as well as other CNS afflictions, according to one embodiment.

FIGS. 2, 3, and 4 are illustrations of various swabs useful for implementing the system for diagnosing a concussion, as well as other CNS afflictions, according to one embodiment.

FIGS. 5 and 6 are illustrations of a swab used in the process for diagnosing a concussion, as well as other CNS afflictions, according to one embodiment.

FIGS. 7 and 8 are illustrations of a tube used in the process for diagnosing a concussion, as well as other CNS afflictions, according to one embodiment.

FIGS. 9, 10 11, and 12 are illustrations of a swab in various stages of the process for diagnosing a concussion, as well as other CNS afflictions, according to one embodiment.

FIG. 13 is a flowchart showing the control flow of an alternative process 1300 for diagnosing a concussion, as well as other CNS afflictions, according to one embodiment.

FIGS. 14, 15, 16, 17, 18, and 19 are illustrations of a nitrocellulose membrane in various stages of the process for diagnosing a concussion, as well as other CNS afflictions, according to one embodiment.

### DETAILED DESCRIPTION

The disclosed embodiments are directed to a rapid, inexpensive and easy-to-use diagnostic test for a variety of central nervous system afflictions, including concussions, among other things. The disclosed embodiments improve over the prior art by providing a diagnostic kit that cannot be falsely interpreted, does not require testing equipment, does not require a trip to a hospital or testing laboratory, is non-invasive and can be easily deployed at a sports field, school, workplace or battlefield. The disclosed embodiments further improve over the prior art by providing a diagnostic kit that provides a definitive result regarding concussions (among other central nervous system afflictions), regardless of the lack of universal consensus regarding the definition of a concussion, as well as other central nervous system afflictions.

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions or modifications may be made to the elements illustrated in the drawings, and the methods described herein may be modified by substituting reordering, or adding additional stages or components to the disclosed methods and devices. Accordingly, the following detailed description does not limit the disclosed embodiments. Instead, the proper scope of the disclosed embodiments is defined by the appended claims.

The Tau Protein

The method and system for diagnosing a concussion (as well as other CNS afflictions) without invasive techniques or complicated wet-laboratory equipment is disclosed herein. The methods utilize the rapid detection of the tau protein which is released in high concentrations into the Cerebrospinal Fluid (CSF) surrounding the brain upon brain injury, including concussion. The insult causes micro-tears in the Brain-Blood Barrier (BBB) causing CSF to leak from the brain into the nasal or auditory passages. This fluid can be collected and utilized as the test material for the concussion diagnostic and even milder concussive events. In addition, this diagnostic kit can be used to diagnose bacterial meningitis, Amyotrophic Lateral Sclerosis (ALS) and clinically active multiple sclerosis, all of which have demonstrated to have elevated tau protein levels in the CSF.

Tau is a microtubule-associated protein localized in neuronal cells and functions as a major structural element in the axonal cytoskeleton. Total tau is abundant in the central nervous system. The hyperphosphorylation of tau is associated with several neurodegenerative diseases that are referred to as tauopathies. Tau levels also markedly influence the pathophysiology of TBI and can serve as an informative biomarker for TBI, including concussion.

After TBI, tau is proteolytically cleaved and gains access to the CSF. In one study CSF levels of tau were significantly elevated in TBI patients compared with control patients, and these levels correlated with clinical outcomes. Several studies have consistently demonstrated that tau CSF levels, which have been closely linked with the presence of axonal injury, increased intracranial pressure, and clinical outcome, are increased in TBI and concussion patients as compared to normal controls. And it has been demonstrated that there is a rapid rise in concentration of tau in CSF following TBI. A common occurrence associated with concussion or TBI is CSF rhinorrhea or the leakage of CSF from the nasal cavity following an intracranial insult. Normally CSF is confined to the space around the brain and spinal cord. Due to its proximity to the sinus and nasal cavity any damage to the Brain-Blood Barrier (BBB), which occurs in concussion will cause the fluid and tau protein to leak and drain out through the nose. Sometimes, instead of the nose, it can leak through the ears where it is known as CSF otorrhea.

CSF has several important functions, such as acting as a shock absorber and thereby protecting the brain and spinal cord during impact, keeping the brain afloat within the cranial cavity, and draining away large proteins and other substances that are not carried out by the vasculature. This most obvious symptom is quite frequently missed by the patient as mistakenly believed to be nasal mucus (runny nose).

In addition, the BBB hinders the assessment of biochemical changes in the brain by use of biomarkers in the blood making the CSF a much more ideal target. However, the BBB integrity can become compromised which can result in an increase in the levels of brain specific proteins in the blood. Some of these biomarkers also get proteolytically degradation in the blood and their levels might be affected by clearance from the blood via the liver or kidney. Nevertheless, the literature on potential peripheral blood biomarkers of brain injury patients with TBI is abundant. Even with these limitations, serum tau levels have recently been demonstrated to rapidly increase following mild TBI and declined after 6 hours post insult. The tau protein levels were also severity-dependent at 1 and 6 hours after TBI. These levels were higher in the severe TBI group than in the mild TBI at 1 and 6 hours.

Neurodegenerative Disorders

Many neurodegenerative disorders share a common pathophysiological pathway involving axonal degeneration despite different etiological triggers. Analysis of cytoskeletal markers like tau in CSF is a useful approach to detect the process of axonal damage and its severity during disease course.

Multiple Sclerosis (MS) is the most common autoimmune disease of the central nervous system in young adults affecting about 30 in 100,000. The majority of MS-patients face the relapsing remitting form of the disease, in which the attacks are usually a sign of acute exacerbation of the inflammation. After an average of 19.1-21.4 years, about one third of the patient's progress to the secondary phase of the disease, which is characterized by slowly accumulating disability with or without acute exacerbations. However, about 11%-18% of the patients have primary progressive multiple sclerosis (PPMS) with continuous slowly accumulating disability.

The pathological hallmark of MS is inflammation induced demyelination and subsequent axonal loss, which may be initially accompanied by re-myelination in part of the lesions. Pathological studies revealed different types of plaques depending on the stage of the inflammatory reaction (active plaques, slowly expanding lesions, inactive plaques and re-myelinated shadow plaques). Histopathological examination of acute MS lesions revealed different patterns of tissue injury indicating possible different mechanisms of the disease cause. T cell infiltrates and macrophage-associated tissue injury (pattern 1); antibody and complement-mediated immune reactions against cells of the oligodendrocyte lineage and myelin (pattern 2); hypoxia-like injury, resulting either from inflammation-induced vascular damage or macrophage toxins that impair mitochondrial function (pattern 3); and a genetic defect or polymorphism resulting in primary susceptibility of the oligodendrocytes to immune injury (pattern 4).

It has been demonstrated that tau protein is highly elevated in CSF in primary multiple sclerosis compared to individuals without MS. Therefore, the disclosed embodiments can be used in the diagnosis of primary MS. CSF can be isolated from the potential MS patient by a routine spinal tap and applied to either tau protein diagnostic kit. Since the tau protein has been elevated to detectable levels in the CSF in MS patients, a positive test result will be a rapid method to indicate high levels of tau in the CSF which will be an indication that the individual is suffering from MS. When used in conjunction with the other battery of MS diagnostics, this embodiment can serve as a very powerful tool to aid in the diagnosis of MS.

Since the first report of ALS more than 100 years ago, the main pathophysiological mechanisms still remain unclear. Degeneration of the motor neurons is followed by an inflammatory reaction with gliosis and accumulation of activated microglia and astrocytes with the production of cytotoxic molecules and inflammatory cytokines like TNF-α and IL-1β. Glial cells also play an important role in the pathophysiology of ALS. Due to a deficient astrocyte-specific glutamate transporter (GLT-1) or excitatory amino acid transporter-2 (EAAT2), the astrocytes fail to clear up the glutamate leading to exacerbation of the glutamine excitotoxicity. Moreover, the role of astrocytes and microglia is supported by the observed increase in production of reactive oxygen species (ROS), nitric oxide and interferon-Y. The role of microglia is more evident in the late stages on the disease.

As far as the disclosed embodiment's functionality in diagnosing ALS, some studies have demonstrated higher levels of tau compared to non-ALS patients. Studies have also demonstrated that patients in the earlier disease stages exhibit a higher level of tau than those with the advanced disease. Since the tau protein has been elevated to detectable levels in the CSF in ALS patients, a positive test result will be a rapid method to indicate high levels of tau in the CSF which will be an indication that the individual is suffering from ALS. When used in conjunction with the other battery of ALS diagnostics, this embodiment can serve as a very powerful tool to aid in the diagnosis of ALS.

Bacterial meningitis is a devastating infection associated with high mortality and morbidity particularly in the neonatal population. Prompt diagnosis and treatment are essential to achieving good outcomes in affected individuals. While the overall incidence and mortality have declined over the last several decades, morbidity associated with neonatal meningitis remains virtually unchanged.

Meningitis is a syndrome classically characterized by some combination of neck stiffness, headache, fever and altered mental status; other symptoms including nausea, vomiting and photophobia are frequently observed as well. Mortality may vary widely according to cause and setting with rates of 3-30% for bacterial meningitis depending on the organism. Aseptic meningitis (usually referring to viral meningitis but also encompassing other “culture-negative” types of meningitis) is generally considered a benign, self-limited disease with low mortality.

CT and MRI may be considered as adjunctive diagnostic tests but are generally nonspecific and show meningeal enhancement. Imaging may be helpful in cases of focal neurologic deficits, particularly when a tuberculoma or cryptococcoma is suspected. Standard diagnostic testing of CSF includes: white blood cell count with differential, total protein, and CSF/blood glucose, used in conjunction with patient history and epidemiology to support potential diagnosis. Total protein and WBC counts reflect inflammation in the CSF while decreased glucose CSF/blood ratio is a sign of glucose consumption by an active infection. These common laboratory tests cannot be the lone laboratory method of diagnosis and while overlap in their values among different diagnoses does occur, general trends emerge and are useful as they help the clinician to focus on particular possible diagnoses. Importantly, up to 40% of persons with Cryptococcus may have an unremarkable CSF WBC count which can mistakenly delay the diagnosis.

Cost remains a significant barrier for many new molecular diagnostics both in high-income and low/middle income countries. Excluding reference laboratories, most local hospital microbiology labs are costs to a healthcare system, not a revenue generator. A new relatively expensive assay may cost more for a microbiology laboratory. A second ironic barrier to adoption is the standard Good Clinical Lab Practice of every laboratory internally validating a new assay. For fully automated U.S. FDA-approved molecular assays, this slows adoption for relatively rare diseases where validation takes significant time and effort. Third, as new molecular tests become available, how best to utilize such testing in a cost-effective manner in high- and low-income settings needs to be explored. Based on these limitations, there is an urgent need for a rapid, inexpensive diagnostic tool for meningitis.

Patients with meningitis are often difficult to classify into bacterial or benign viral meningitis. On admission, patients with bacterial meningitis do not always display the typical clinical signs and laboratory findings can be confounding. CSF leukocyte count in bacterial meningitis can be lower than 100/ul indicating a sever course. CSF leukocyte count differentiation is also imprecise. More than 30% of the bacterial meningitis patients with CSF leukocyte counts less than 1,000/ul display a CSF lymphocytosis instead of the typical granulocytosis.

Facing these difficulties, a bacterial meningitis score was designed. While this score allowed the identification of all but two of more than 121 pediatric bacterial meningitis patients, too many viral meningitis patients showed scores that were indicative of bacterial meningitis. Similarly, Gram stain, the occurrence of seizure at or before presentation, peripheral leukocyte count, CSF leukocytes and protein CSF concentration could not correctly classify all pediatric bacterial meningitis patients. Of those who were, according to this score, at risk for bacterial meningitis, more than 40% and to be reclassified as viral meningitis. A score built of C-reactive protein and protein CSF content falsely classified as many as 16 of 71 pediatric patients with viral meningitis.

CSF leakage renders one more susceptible to infections of the brain such as subdural or epidural infections due to Neiseeria meningitis, Staphylococcus pneumonia or Staphylococcus aureus. On the other hand, meningitis infections can compromise the dura resulting in CSF leakage. Additionally, CNS neoplasms and or abscesses within the brain can also result in a compromised dura and blood brain barrier and may cause leakage of CSF. Therefore, the disclosed embodiments could be used to collect the CSF fluid that has been leaked though the BBB and be used to detect the tau protein thereby acting as a preliminary indicator for the above said conditions.

Tau protein has been documented to be elevated in both bacterial meningitis and encephalitis, making tau protein an ideal target for a biomarker for bacterial meningitis. In addition, this makes the disclosed embodiments an ideal candidate as a diagnostic tool for bacterial meningitis. Since a rapid, accurate diagnosis of bacterial meningitis is paramount to treatment and the survivability of bacterial meningitis, the rapid diagnosis using the disclosed embodiments will help differentiate bacterial from viral meningitis leading to a better outcome for the individual suffering from the more lethal form, bacterial meningitis.

CSF can be isolated from the afflicted individual and applied to the disclosed embodiments. If the tau protein is detected in the CSF, this would be a clear indication that the individual is suffering from bacterial meningitis where CSF tau protein levels are elevated to detectable levels compared to low levels of tau in viral meningitis. Any of the disclosed diagnostic kit designs can be used, and ideally the CSF should be used by a routine spinal tap as the source material for the diagnostic kit. Taken together with the other symptoms that the patient displays, medical history and other battery of bacterial meningitis diagnostics, this embodiment can serve as a powerful tool to assist in the diagnosis of bacterial meningitis.

The Diagnostic Kit

The method and system for diagnosing a concussion (as well as other CNS afflictions) without invasive techniques or complicated wet-laboratory equipment comprises a diagnostic kit that includes a color-changing swab 502 and a tube 702. In a first embodiment, the diagnostic kit detects the tau protein rapidly by enabling the swab to change color, indicating the presence of CSF.

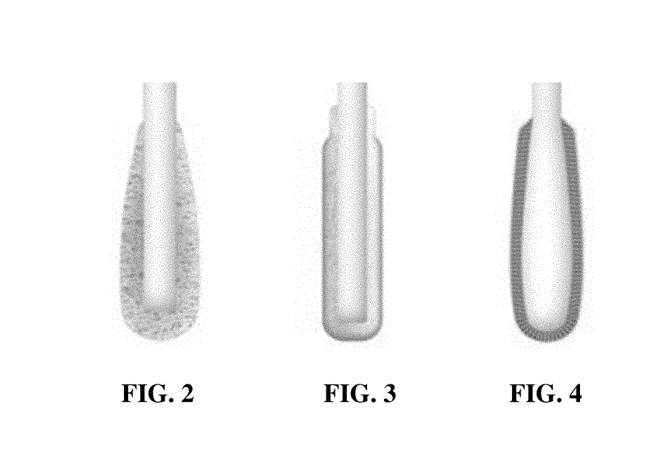

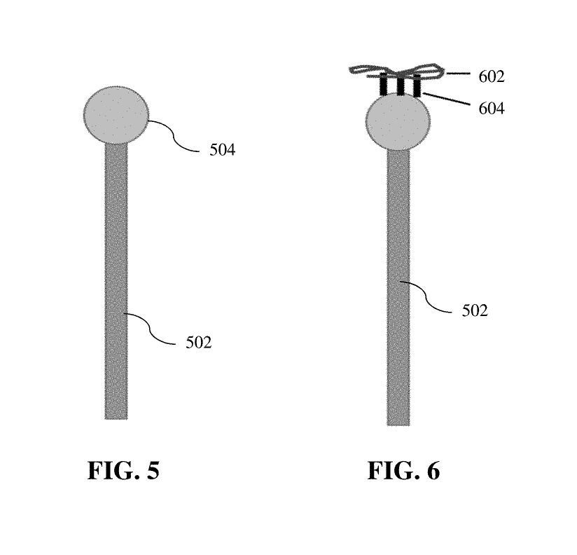

The method may employ a polyurethane, foam-tipped hydrophobic nasopharyngeal swab (see FIG. 3) for nasal collection to collect CSF that has been discharged from the nose or from the ears following insult. A collar may be added at 5.5 cm as a guide for maximum insertion depth. For ear collection, a flocked hydrophobic ear swab (see FIG. 4) or a fiber-tipped swab (see FIG. 2) may be employed, inserted gently into the ear and rotated to collect the discharged CSF. The swab may be hydrophobic foam so that the tau protein remains bound.

The swab containing the CSF will have the target biomarker, tau, which, after insult, is typically found in elevated concentrations in the CSF and is not found in the nasal passage nor the ear canal. Tau is specifically housed in neuronal tissue and CSF. The only way that tau will be detected will be due to increased concentrations of tau due to intracranial insult and due to leakage of CSF caused by intracranial insult. The method employs buffers where the salt concentration in the tube 702 is below 750 mM (milli-molars) which will not force any secondary structure formation of the tau protein. The buffers will also be devoid of any polyanions which can induce the aggregation of tau through the masking of charged amino acids in the tau protein potentially leading to the masking of the epitope and failure of the antibody binding. The method also employs an antibody or antibodies that are designed to bind irreversibly to the target concussion biomarker tau (i.e., a tau-specific antibody). The antibody/antibodies will have a colorimetric color change indicator that will change color based on the presence of tau.

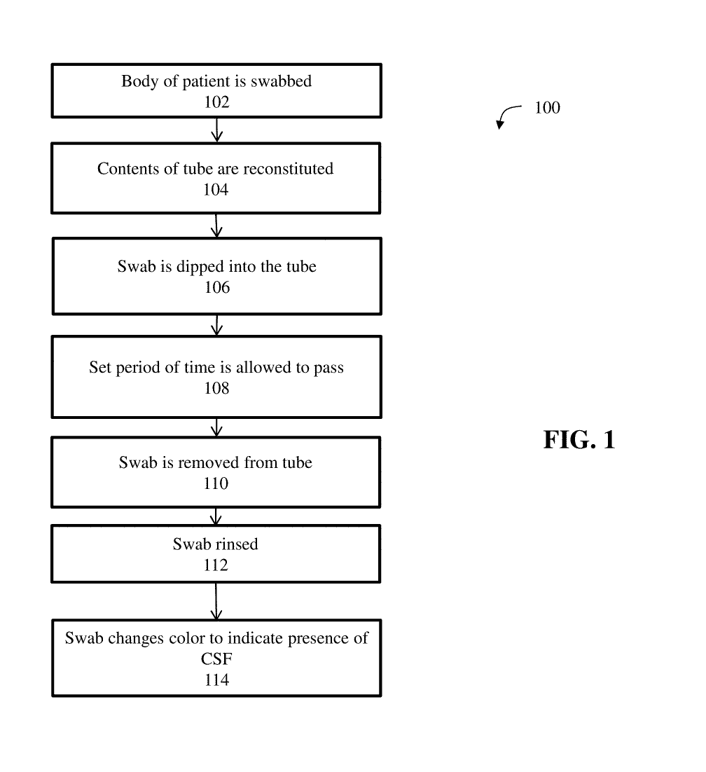

FIG. 1 is a flowchart showing the control flow of the process 100 for diagnosing a concussion, as well as other CNS afflictions, according to one embodiment. This detection method exploits a unique biochemical feature of the tau protein. Tau, unlike the majority of proteins in the body is an intrinsically un-folded protein. Tau does not assume a well-structured secondary or tertiary structure. Because of this, tau has its hydrophobic amino acids exposed to solution, which is very uncommon, biochemically.

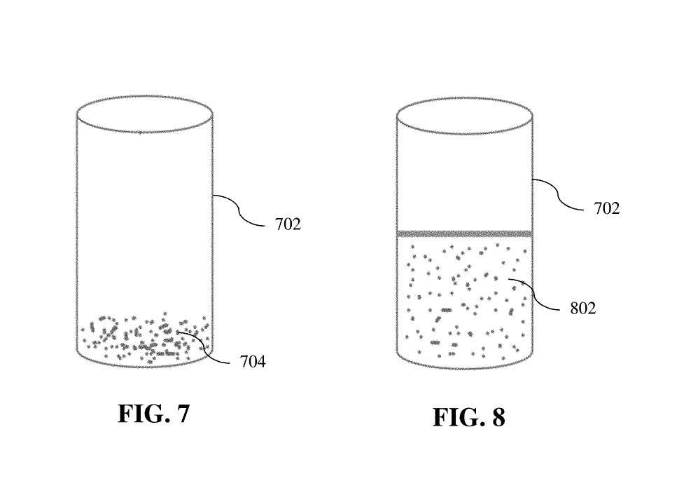

The process of FIG. 1 begins with a step 102 wherein a portion of the body of the patient (ear canal, nasal cavity, etc.) is swabbed with the swab 502. The swab will have a hydrophobic tip 504. The hydrophobic swab tip will bind to the hydrophobic amino acids exposed on the tau protein 602 in the CSF, resulting in a very tight and stable hydrophobic-hydrophobic interaction 604 between the swab 504 and tau protein 602 (see FIG. 6). The tube 702 of the kit will contain a lyophilized tau-specific antibody 704 conjugated to 400 nm red latex nanoparticles (see FIG. 7). Alternatively, beads of about 2.8 μm may be used. The red latex conjugate will serve as the color indicator for a positive test result. The tube 702 of the kit may contain 0.5 mg of the lyophilized tau-specific antibody 704. In this document, the term conjugated refers to linking, bonding, coupling, covalently coupling or a compound formed by the joining of two or more chemical compounds.

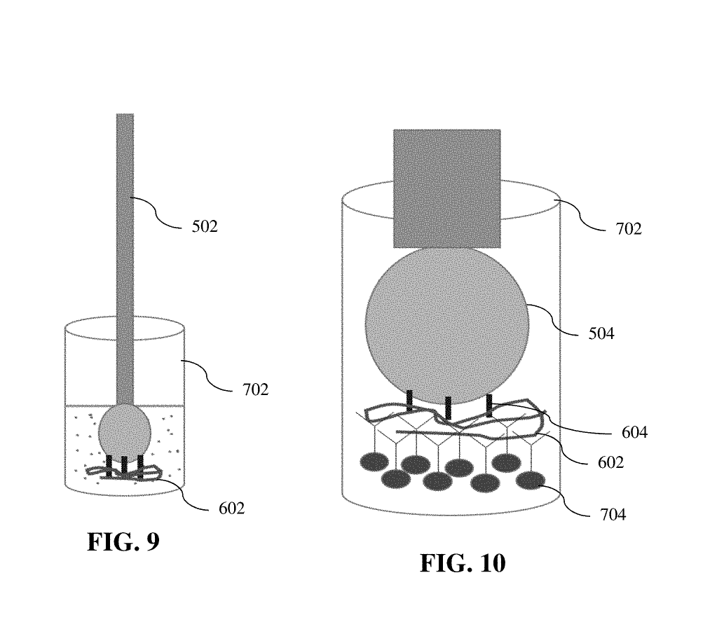

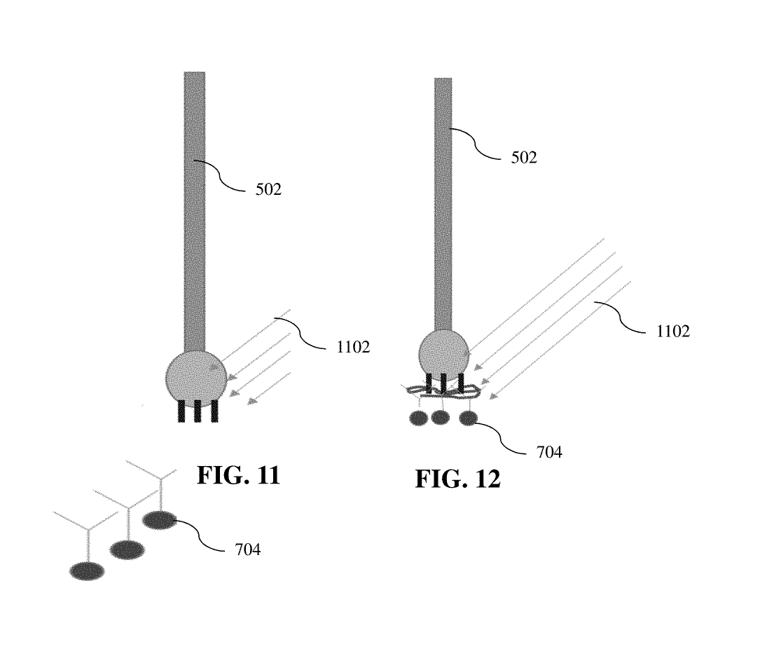

In step 104, the lyophilized antibody 704 will be reconstituted with about 3 mL (for example) of phosphate-buffered saline (PBS), which is contained in the diagnostic kit (see FIG. 8). In step 106, the collection swab that has been used to collect the CSF and tau protein will be placed into the plastic tube 704 (see FIG. 9) containing the tau-specific antibody conjugated to the red latex nanoparticles and, in step 108, allowed to incubate for a set period of time, such as 15 minutes. During this time, the lyophilized tau-specific antibody 704 reacts with the tau protein 602 in the CSF on the swab 502 (see FIG. 10).

After the incubation period, the swab 502 is removed from the tube in step 110. In step 112, the swab 502 may be rinsed thoroughly with distilled water 1102, which is contained in the diagnostic kit. If the diagnostic is negative for concussion, then the rinsing will remove the lyophilized antibody 704 and there will be no red color change to the swab 502 (see FIG. 11). If the diagnostic is positive (meaning the tau protein is on the collection swab and has reacted with the tau-specific antibody), then the rinsing will not remove the lyophilized antibody 704 because the antibody-antigen interaction will prevent the latex labelled antibody 702 from being washed away (see FIG. 12). In this case, in step 114 there will be a red color change to the swab tip 504, indicating a positive test result for concussion.

If the diagnostic is negative for concussion, then there will be no red color change to the swab 502. If the diagnostic is positive (meaning the tau protein is on the collection swab and has reacted with the tau-specific antibody), then in step 112 there will be a red color change to the swab tip 504 indicating a positive test result for concussion.

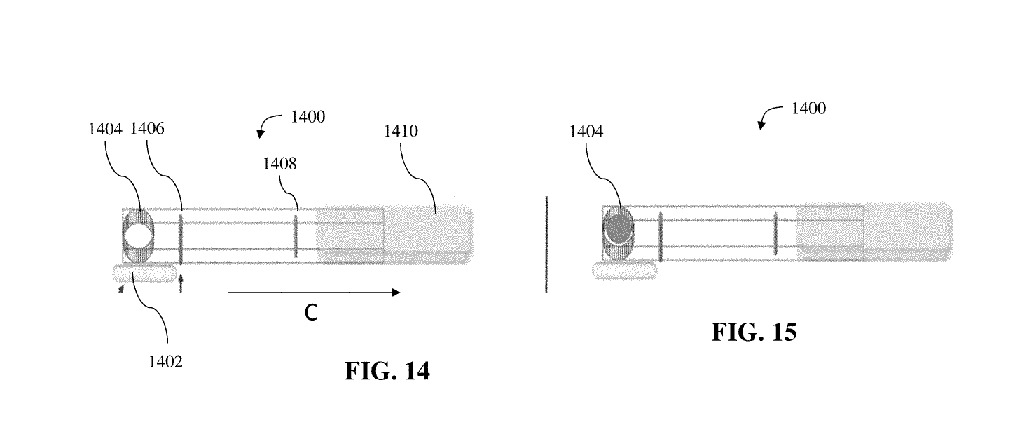

A second disclosed embodiment for diagnosing a concussion, as well as other CNS afflictions, is disclosed below. This second detection method employs a rapid lateral flow capture assay (or test) 1400 (see FIG. 14) to detect the presence of tau in discharged CSF. Lateral flow tests, also known as lateral flow immunochromatographic assays, are simple devices intended to detect the presence (or absence) of a target analyte in sample (matrix) without the need for specialized and costly equipment. Typically, these tests are used for medical diagnostics either for home testing, point of care testing, or laboratory use. A widely spread and well known application is the home pregnancy test.

The second disclosed method also uses a non-hydrophobic nylon-flocked nasopharyngeal diagnostic swab to capture discharged CSF from concussed individuals. The swab may be equipped with a collar at 5.5 cm as a guide to maximum insertion depth. The swab should not be hydrophobic (but rather hydrophilic) so that the tau protein is transferred to the nitrocellulose membrane and does not remain bound to the swab. Since a large volume of fluid is required for this second test, the flocked swab (see FIG. 4), which absorbs 3 times the fluid volume, is the preferred collection method for this second test. For ear collection, the second method uses a non-hydrophobic ear diagnostic collection swab, which is gently inserted into the ear and rotated to collect any discharged CSF.

The second method employs specific IgG anti-tau antibody at a specific concentration conjugated to 400 nm red latex particles attached directly to a high flow nitrocellulose membrane (e.g., lateral flow test) (see item 1400 in FIG. 14) at a sample loading area where the nitrocellulose membrane binds proteins electrostatically through interactions of the strong dipole of the nitrate ester with the strong dipole of the peptide bond of the protein, wherein the interaction is completely independent of the pH. The latex particles will be attached to the Fc region of the antibody (fragment crystallizable region), the constant region of the antibody, and the region that does not bind to the epitope, in this case, tau. The latex particles serve as the visual indicator that the tau protein is present. In short, if the tau protein is present in sufficient concentration, the anti-tau antibody will bind to the tau protein in the CSF. Since this antibody has red latex nanoparticles attached to it, it will allow the visualization of the tau protein though the red color change. The second method may also include antibodies that are affinity-purified, and may employ a nitrocellulose membrane with a specific pore size and has a specific capillary flow rate. Further, the nitrocellulose membrane may have a specific overall total length.

The overall total length of the membrane must meet certain criteria. If the nitrocellulose membrane is too long, sample diffusion will occur and hence decreased target sample concentration could prevent the detection of tau. If the length of the nitrocellulose membrane is too short, it will have a negative effect on the resolution of the color indicator, making it blurry and uneven.

The second method also employs a 2nd anti-tau antibody (item 1408 in FIG. 14) downstream from the 1st anti-tau antibody (see item 1406 in FIG. 14) and functions as the capture line. The 2nd anti-tau antibody will bind to a different region of the tau protein, since the first antibody will have already masked the first epitope (binding site) because the first antibody will be bound there. The second method may also employ a cellulose absorbent cloth (see item 1410 of FIG. 14) downstream of the capture line to increase the lateral flow (see arrow C of FIG. 14) of samples and antibodies. In essence the absorbent cloth will help “pull” the sample through the nitrocellulose membrane.

The second method further employs a bed (see item 1410 in FIG. 14) for the filter paper of cellulose filter paper, binding buffers that are devoid of Tween 20 and Triton X-100 (both chaotropic agents that can physically interfere with the antibody protein and nitrocellulose interactions—thus, if these compounds are used, then they should be used at concentrations lower than 0.01% v/v), due the inhibitory binding activity of these compounds, and a specific membrane whose lateral flow rate is specific as a function of antibody-antigen complex formation where: R=k[AB][Ag]. Wherein the amount of antibody-antigen complex formed, R, is equal to k, a rate constant related to the affinity of the antibody for the antigen, times the concentrations of the reactants AB (antibody) and Ag (antigen). Thus, at a flow rate of 1x: R=k[AB][AG].

Enabling the correct capillary flow rate is important because the effective concentration of an analyte in the sample (tau) is inversely proportional to the square of the change in flow rate. In a lateral flow test, the antigen is unable to bind once it passes the immobilized antibody because the test is designed to flow in only one direction. As a consequence of the test design, the effective antigen concentration decreases with the square of the increase in flow rate because of the reduced length of time that the components of the reactive pair (antibody and antigen) are close enough to bind to each other. Further, doubling the flow rate effectively decreases the concentration of the complex by 4-fold which would make the detection of the complex formation very difficult: R=k[0.5×Ab][0.5×Ag]=0.25 [Ab][Ag]. If the flow rate doubles, each component is only close enough for half the amount of time. Thus, if the flow rate doubles, [AB] and [AG] don't actually change; their effective concentrations change because they spend only half as much time in close proximity to bind each other.

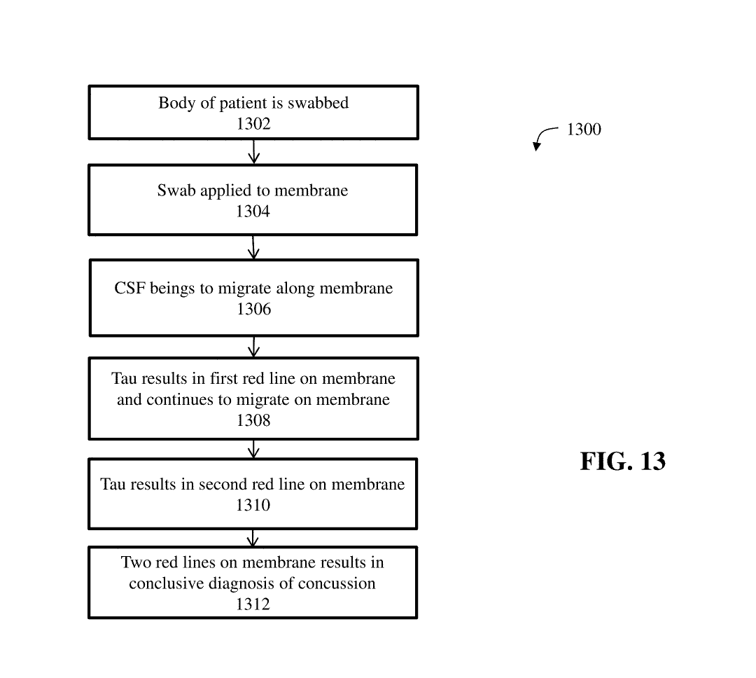

The process of FIG. 13 begins with a step 1302 wherein a portion of the body of the patient (ear canal, nasal cavity, etc.) is swabbed with the swab 502. Discharged CSF will be collected by the non-hydrophobic swab. This discharged CSF is the result of the insult to the head and will contain the tau protein. In the event that one does not perform swabbing of the nasal passage or ear canal within an hour post-injury, a spray bottle containing sterile saline is included in the kit. Slight irrigation of nose or ear will facilitate capture of the tau protein on the swab and facilitate optimal transfer to the membrane.

In step 1304, the fluid on the swab will then be transferred onto the sample application area 1404 (see FIG. 14) of the concussion diagnostic nitrocellulose membrane, such as via sample pad 1402. If the amount of CSF fluid is small, a small squirt bottle will be available to mix the CSF fluid into and that fluid (Tris buffer solution, for example) will be used to apply the CSF directly onto the sample application area 1404 of the concussion diagnostic membrane, or sample pad 1402.

The concussion diagnostic membrane will have already contain a line 1406 of an IgG anti-tau antibody conjugated to 400 nM, for example, of red latex beads already applied and dried on the membrane. This antibody will be applied to the membrane without the use of chaotrophic agents, which could potentially physically disrupt the interactions of the antibody to the membrane.

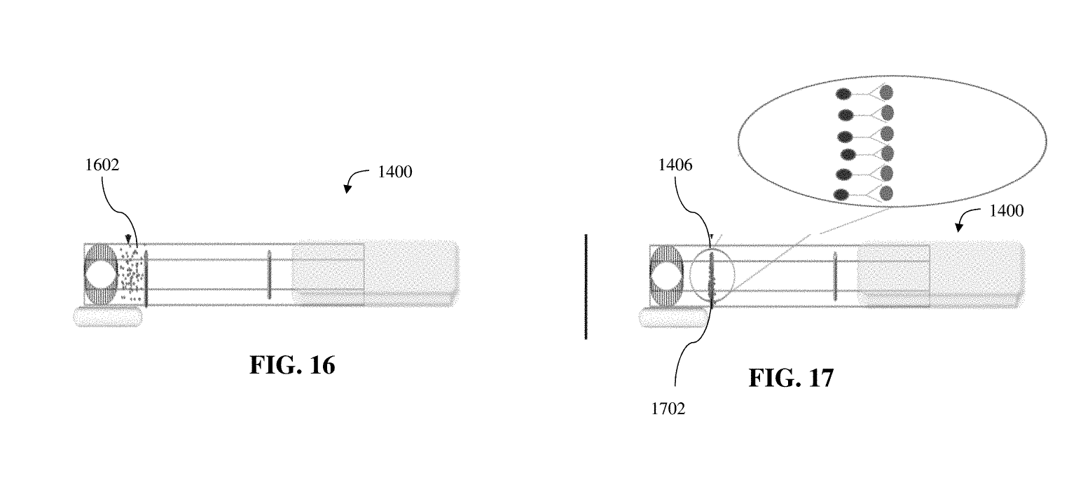

After the application of the CSF to the sample application area 1404 of the concussion diagnostic membrane, in step 1306, the CSF fluid will begin to migrate via lateral flow (capillary action—direction of arrow C in FIG. 14)) down the membrane towards the target capture line 1406. FIG. 16 show the CSF 1602 travelling towards 1406. The target capture line 1406 consists of a tau-specific antibody conjugated to 400 nm red latex beads.

Once tau comes into contact with the target line 1406, in step 1308, the tau-specific antibody conjugated to the 400 nM red latex beads will bind to the tau protein and continue migrating with the protein down the membrane. Tau binds to the latex conjugated antibody and carries the antibody along with it during lateral flow. Not all of the latex antibody will migrate with the tau, however, leaving some behind, and therefore leaving an initial red line at 1406.

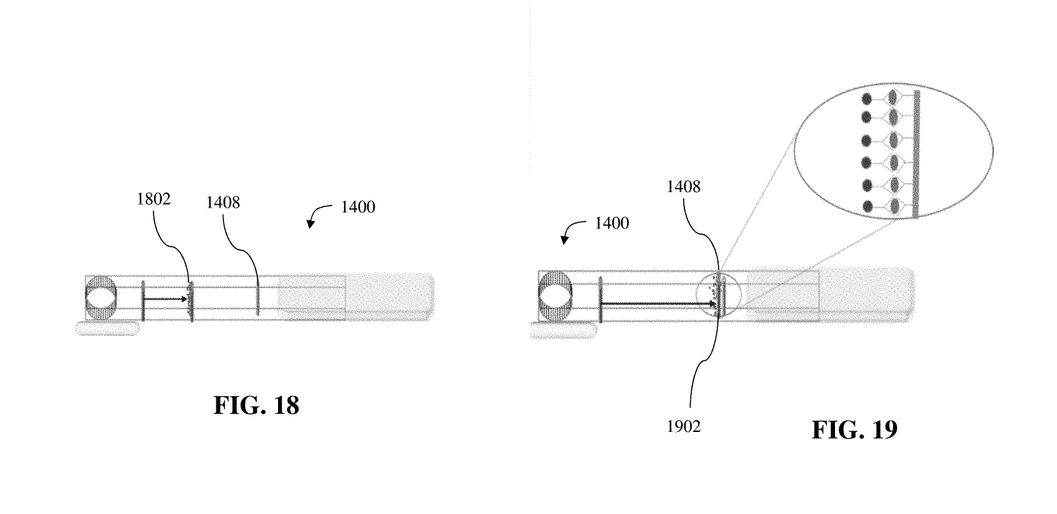

FIG. 17 shows that the CSF 1702 has reached line 1406. If tau is present, it will bind irreversibly to the anti-tau antibody conjugated to the red latex nanoparticles and form a tau-antibody-red latex complex at line 1406. FIG. 18 shows that the tau and anti-tau latex conjugated antibody 1702 has passed line 1406 and continues to migrate through the nitrocellulose membrane towards line 1408.

The tau protein-tau antibody conjugated to the 400 nM latex nanoparticles will migrate until it comes in contact with the second capture line 1408. The capture line 1408 also consists of a tau specific antibody, IgG, which is not conjugated and binds to a different epitope of the tau protein. Line 1408 will “capture” the initial tau-anti-tau latex conjugated antibody and create a 2^{nd} red line on the concussion diagnostic membrane in step 1310. The tau-anti-tau conjugated to latex antibody is then captured by the second unconjugated tau antibody immobilized on the membrane. FIG. 19 shows that the CSF 1902 has reached line 1408.

If tau is present, which will be the indicator of this diagnostic, it will be captured by the unconjugated antibody 1408 and the result will be the presence of a 2nd red line on the nitrocellulose membrane at 1408. If tau is not present, there will not be the formation of a second red line at 1408 thereby yielding a negative result on the concussion diagnostic. Thus, the presence of tau will result in two red lines 1406 and 1408, but the lack of tau will not result in two red lines. Consequently, two red lines 1406 and 1408 in the concussion diagnostic membrane results in a conclusive diagnosis of a concussion in step 1312.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A diagnostic kit comprising:
a tube containing a predefined amount of lyophilized tau-specific antibody conjugated to colored latex nanoparticles;
a fluid for mixing with the lyophilized tau-specific antibody conjugated to colored latex nanoparticles within the tube, so as to reconstitute the lyophilized tau-specific antibody conjugated to colored latex nanoparticles, resulting in a reconstituted mixture; and
a swab comprising an absorbent hydrophobic material, the swab configured for absorbing cerebrospinal fluid when swabbed on a patient, wherein when the swab that has absorbed cerebrospinal fluid is placed in the tube containing the reconstituted mixture, the swab is configured to change color.

2. The diagnostic kit of claim 1, wherein the swab includes a swab tip configured for binding to hydrophobic amino acids in tau protein in cerebrospinal fluid of the patient.

3. The diagnostic kit of claim 2, wherein the lyophilized tau-specific antibody in the tube comprises about 0.5 mg of lyophilized tau-specific antibody.

4. The diagnostic kit of claim 3, wherein the colored latex nanoparticles are 400 nm red latex nanoparticles.

5. The diagnostic kit of claim 4, wherein the fluid for mixing with the lyophilized tau-specific antibody conjugated to colored latex nanoparticles within the tube comprises about 3 ml of phosphate-buffered saline.

6. A method for diagnosing concussions and other central nervous system disorders, the method comprising:
swabbing a patient with a swab comprising an absorbent hydrophobic material, so as to absorb cerebrospinal fluid of the patient into the swab;
pouring a fluid into a tube containing a predefined amount of lyophilized tau-specific antibody conjugated to colored latex nanoparticles, so as to reconstitute the lyophilized tau-specific antibody conjugated to colored latex nanoparticles, resulting in a reconstituted mixture;
placing the swab in the tube containing the reconstituted mixture for a predefined period of time;
removing the swab from the tube and rinsing with water; and
observing a change of color of the swab, wherein the swab is configured to change color when cerebrospinal fluid of the patient is absorbed into the swab.

7. The method of claim 6, wherein the step of swabbing a patient further comprises:
swabbing a patient with a swab comprising an absorbent hydrophobic material, so as to absorb cerebrospinal fluid of the patient into the swab, wherein the swab includes a swab tip configured for binding to hydrophobic amino acids in tau protein in cerebrospinal fluid of the patient.

8. The method of claim 7, wherein said predefined amount of lyophilized tau-specific antibody conjugated to colored latex nanoparticles comprises about 0.5 mg of lyophilized tau-specific antibody.

9. The method of claim 8, wherein said the colored latex nanoparticles comprise about 400 nm red latex nanoparticles.

10. The method of claim 9, wherein said fluid comprises about 3 ml of phosphate-buffered saline.

11. The method of claim 10, wherein said predefined period of time comprises about 15 minutes.

12. A diagnostic kit comprising:
a swab comprising an absorbent non-hydrophobic flocked swab, the swab configured for absorbing cerebrospinal fluid when swabbed on a patient;
a high flow nitrocellulose membrane configured for lateral flow in a first direction via capillary action;
a sample loading area located at a first end of the membrane and upstream of the first direction;
a first set of anti-tau antibodies conjugated to colored latex particles located at a first location on the membrane and downstream of the sample loading area;
a second set of anti-tau unconjugated antibodies located at a second location on the membrane and downstream of the first location; and
a cellulose absorbent cloth located at a second end of the membrane and downstream of the first direction, so as to aid the lateral flow;
wherein when the sample loading area has absorbed cerebrospinal fluid transferred from the swab, the first and second locations on the membrane are configured to change color.

13. The diagnostic kit of claim 12, wherein the swab includes a flocked swab tip.

14. The diagnostic kit of claim 13, wherein the colored latex nanoparticles comprise red latex nanoparticles.

15. The diagnostic kit of claim 13, wherein the antibody is unconjugated.

16. The diagnostic kit of claim 13, wherein the first set of anti-tau antibodies comprise IgG anti-tau antibodies.

17. The diagnostic kit of claim 13, wherein the second set of anti-tau antibodies comprise IgG anti-tau antibodies.
